(19) United States
(12) Patent Application Publication
(10) Pub. No.: US 2022/0127681 A1
(43) Pub. Date: Apr. 28, 2022

(54) SORTING CELL-TYPE SPECIFIC EXTRACELLULAR VESICLES

(71) Applicant: THE TRUSTEES OF COLUMBIA UNIVERSITY IN THE CITY OF NEW YORK, New York, NY (US)

(72) Inventors: Gordana VUNJAK-NOVAKOVIC, New York, NY (US); Bohao LIU, New York, NY (US); Bryan WANG, Morganville, NJ (US)

(21) Appl. No.: 17/434,558

(22) PCT Filed: Feb. 27, 2020
(86) PCT No.: PCT/US20/20126

§ 371 Date: Aug. 27, 2021

Related U.S. Application Data
(60) Provisional application No. 62/811,736, filed on Feb. 28, 2019.

(51) Int. Cl. C12Q 1/6886; C12Q 1/6809; C12Q 1/6806; C12N 5/077; G01N 33/50; C12Q 1/6841

(52) U.S. Cl. CPC C12Q 1/6886; C12Q 1/6809; C12Q 1/6806; C12N 5/0657; C12Q 2600/112; C12Q 1/6841; C12Q 2600/158; C12Q 2600/178; C12N 2506/45; G01N 33/5023

(57) ABSTRACT

Disclosed is a method for identifying cell-type specific EV markers comprising culturing induced pluripotent stem cells (iPSCs) to provide a culture of a first differentiated cell type; isolating extracellular vesicles (EV)s from the culture of the differentiated cell type; generating a microscopic RNA (miRNA) expression profile for the differentiated cell type by next-generation sequencing; and identifying specific miRNA markers associated with the differentiated cells. Also disclosed are methods for in vitro testing for a disease condition comprising providing a biological sample; isolating extracellular vesicles from said sample by labeling the extracellular vesicles using fluorescence in situ hybridization, and sorting the labeled extracellular vesicles by high resolution flow cytometry; and determining the status of exosomes and/or an exosome-associated marker in the sample, wherein an abnormal status of exosomes and/or the exosome-associated marker in the sample indicates the presence of a disease condition.

### CROSS-REFERENCE TO RELATED APPLICATIONS

This application claims priority to U.S. Provisional Application Ser. No. 62/811,736 filed Feb. 28, 2019, the contents of which is incorporated in their entirety herein.

### BACKGROUND OF THE DISCLOSED SUBJECT MATTER

Biomarkers are critical indicators of health and disease. Biomarkers can be used for early disease detection, to direct clinical decision making, to predict outcomes, and to evaluate responses to treatment and intervention. While biomarkers can range in complexity (from temperature and pulse to circulating tumor DNA), most current biomarkers are limited to measurements of a small number of specific and often superficial signals. As diseases involve perturbations of the complex intercellular networks that link tissues and organ systems, current biomarkers do not provide a comprehensive picture of disease processes.

Recently, extracellular vesicles (EVs) have emerged as a promising new class of biomarkers. EVs are small membrane-bound vesicles and are produced by every type of cell in multicellular organisms, and have attracted an increasing amount of research in recent years due to their role in intercellular communication. They generally are produced either from the plasma membrane or an endosome of the cell. EVs include for example but not limitation (i) exosomes: 30-100 nm diameter membraneous vesicles of endocytic origin (ii) ectosomes (also referred to as shedding microvesicles, SMVs): large membranous vesicles (50-1000 nm diameter) that are shed directly from the plasma membrane (PM) and (iii) apoptotic bodies (50-5000 nm diameter): released by dying cells.

Exosomes are natural lipidic extracellular nanovesicles produced and released by virtually all cell types in a finely regulated and functionally relevant manner so that the protein and mRNA composition reflects the type and condition of a parent cell. These vesicles have intrinsic stability and ability to cross biological barriers, so that exosomes originated from different tissues can be found in easily accessible biological fluids such as blood, blood fractions such as plasma or serum, saliva, sputum, ascites, urine, cyst fluid, pleural fluid, peritoneal fluid, cerebrospinal fluid, amniotic fluid, semen or milk.

The contents of an EV varies depending on the type of parent cell and the region of the cell which produced the EV. EVs carry a diverse cargo of bioactive molecules including microRNAs (miRNA) mRNA, DNA, proteins, and lipids. Importantly, the contents of EVs released from a given cell depends not only on the identity of the parent cell, but also on the physiological status of that cell. As the status and behavior of a cell changes over time and in response to various stressors, so too does the content of the cell-secreted EVs.

Thus, extracellular vesicles (e.g., exosomes), or the markers associated with them, are often found in altered status in the samples of subjects having certain diseases or disease-related pathologies compared to samples from healthy individuals. This is especially true of epithelial cancers (e.g., those of the lung, colon, breast, prostate, ovaries, endometrium, etc.), diabetic nephropathy, etc.

Given their biological roles and features, exosomes are considered early sentinels of alterations in cell and tissue homeostasis and metabolism and are an appealing source for identification of novel disease-relevant biomarkers as well as display of known tissue markers in a liquid biopsy paradigm.

As such, many have begun to explore the use of EV biomarkers for diseases ranging from cancer to heart failure. Analysis of the contents of EVs secreted by a certain cell can provide a unique window into the complex, dynamic behaviors of a cell without damaging or affecting the cell in any way. For instance, exosomes are EVs produced via an endocytic pathway, and can contain endosome-associated proteins, such as Rab GTPases or SNAREs.

Existing methods isolate all systemic EVs from a subject biofluid such as plasma, resulting in a heterogeneous mixture of EVs from many cell types across multiple organ systems, making it difficult to determine their cells of origin. While subsequent analysis of systemic EV contents have revealed some markers of disease, the inability to separate EVs based on their cells of origin severely limits our ability to access all the information contained in EVs.

It is desirable to be able to isolate, separate and characterize EVs based on their parent cell type. Characterization of the contents of each subpopulation of cell-type specific EVs could provide unparalleled insight into the pathophysiological status of parent cells. This ability would advance the utility of EVs as biomarkers, allowing them to provide diagnostic information on the pathophysiological status of multiple organ systems. Major challenges lie in the association of exosome-associated markers, such as proteins and RNAs, to a particular tissue, in a particular condition and optimization of reliable, affordable, noninvasive exosome targeted solutions and assays that can be realistically implemented in clinical research and practice.

### SUMMARY OF THE DISCLOSED SUBJECT MATTER

Accordingly, in a first aspect of the disclosed subject matter, there is provided a method for identifying cell-type specific EV markers, the method comprising

culturing induced pluripotent stem cells (iPSCs) to provide a culture comprising a pure population of a first differentiated cell type therefrom;

isolating extracellular vesicles (EV)s from the culture of the differentiated cell type;

generating a microscopic RNA (miRNA) expression profile for the differentiated cell type by next-generation sequencing; and

identifying specific miRNA markers associated with the pure population of the differentiated cell type by differential expression analysis by comparison to a miRNA expression profile of a population of a second differentiated cell type different from the first differentiated cell type.

In an embodiment, the induced pluripotent stem cells (iPSCs) are hybridized with an EV membrane marker CD63 linked to NanoLuc luciferase.

In an embodiment, the first differentiated cells retain the EV membrane marker CD63 linked to NanoLuc luciferase from their parent (iPSCs) hybridized with an EV membrane marker CD63 linked to NanoLuc luciferase.

In an embodiment, the second differentiated cells are labeled with the EV membrane marker CD63 linked to NanoLuc luciferase.

In an embodiment, isolating extracellular vesicles (EV)s from the culture of the differentiated cell type comprises high resolution flow cytometry.

Embodiments of this aspect of the disclosed subject matter include those wherein the induced pluripotent stem cells (iPSCs) are organ specific.

In a particular embodiment, the iPSCs are specific to the cardiovascular system.

In particular embodiments, the first differentiated cells are cardiomyocytes, atrial cardiomyocytes, ventricular cardiomyocytes, cardiac fibroblasts, macrophages or endothelial cells.

In a second aspect of the disclosed subject matter, there is provided a method for determining in vitro the presence of a disease condition in a subject, such method comprising;

a) providing a biological sample obtained from that subject,

b) isolating extracellular vesicles from said sample, wherein this step of isolating extracellular vesicles comprises

1) labeling the extracellular vesicles using fluorescence in situ hybridization, and

2) sorting the labeled extracellular vesicles by high resolution flow cytometry;

c) determining, from the extracellular vesicles isolated in step b), the status of exosomes and/or an exosome-associated marker in a sample obtained from the subject; wherein an abnormal status of exosomes and/or the exosome-associated marker in the sample indicates the presence of a disease condition in the subject.

In some embodiments status is determined by determining the level of an indicator exosome or an exosome-associated marker in the sample, such as by comparing the level of a specific type of exosome, an indicator exosome, with the aggregate level of all exosomes in the sample. This may comprise determining the status (e.g., the level) of an exosome-associated marker. It may also comprise isolating exosomes from the sample and determining the status (e.g., the level) of an exosome-associated marker.

In some embodiments the exosome-associated marker is a cancer-marker, a cancer-type marker, and/or a tissue-type marker. In other embodiments, the exosome-associated marker is a marker indicative of inflammatory disease, infectious disease, an autoimmune disease, hypersensitivity-associated inflammation, graft rejection, injury, disorder of apoptosis or disease associated with activation of the necroptosis activation pathway.

Notably, the indicator exosome or the exosome-associated marker in the sample is characterized by a specific miRNA marker. Such specific miRNA markers can be determined by the method of the first aspect of the disclosed subject matter.

In some embodiments, the method comprises providing a biological sample from a subject; isolating exosomes comprising microRNAs (miRNAs) from the biological sample; determining an amount of one or more of the miRNAs; and comparing the amount of the one or more miRNAs to one or more miRNA control levels. The subject is then diagnosed as having a disease condition if there is a measurable difference in the amount of the one or more miRNAs from the subject's exosomes as compared to the one or more miRNA control levels. In some embodiments, the method further comprises selecting a treatment or modifying a treatment for the disease condition based on the amount of the one or more miRNAs determined.

In an embodiment, the level or presence of a suitable biomarker (e.g. a specific miRNA marker or miRNA expression profile is determined in step c), and

d) the status of exosomes and/or an exosome-associated marker in the sample obtained from the subject is determined by comparing the level or presence of the biomarker determined in step c) with one or more reference values.

In one embodiment, the reference value is the level or presence of the same biomarker of step c) in an earlier sample from the same subject as in step a).

In another embodiment, the reference value is the level or presence of the same biomarker of step c) in samples obtained from a subject different from than the subject of step a).

In some embodiments of the second aspect of the disclosed subject matter, a method for diagnosing a cancer in a subject is provided. In some embodiments, the method comprises providing a biological sample from a subject; isolating cancer-derived exosomes comprising microRNAs (miRNAs) from the biological sample; determining an amount of one or more of the miRNAs; and comparing the amount of the one or more miRNAs to one or more miRNA control levels. The subject is then diagnosed as having the cancer if there is a measurable difference in the amount of the one or more miRNAs from the cancer-derived exosomes as compared to the one or more miRNA control levels. In some embodiments, the method further comprises selecting a treatment or modifying a treatment for the cancer based on the amount of the one or more miRNAs determined.

In other embodiments, a method for evaluating treatment efficacy and/or progression of a cancer in a subject is provided. In some embodiments, the method comprises providing a series of biological samples over a time period from a subject; isolating cancer-derived exosomes comprising miRNAs from the series of biological samples; determining an amount of one or more of the miRNAs in each of the biological samples from the series; and determining any measurable change in the amounts of the one or more miRNAs in each of the biological samples from the series to thereby evaluate treatment efficacy and/or progression of the cancer in the subject.

In an embodiment, the method is used for determining in vitro the tumor transformation status in a subject.

Another embodiment provides a method of detecting recurrence of cancer in a subject, wherein an abnormal status of exosomes and/or the exosome-associated marker in the sample indicates recurrence.

In still other embodiments of the presently-disclosed subject matter, a method for characterizing a cancer in a subject is provided. In some embodiments, the method comprises providing a biological sample from a subject; isolating cancer-derived exosomes comprising miRNAs from the biological sample; determining an amount of one or more of the miRNAs; and comparing the amount of the one or more miRNAs to one or more miRNA control levels. The cancer is then characterized based on a measurable difference in the amount of the one or more miRNAs from the cancer-derived exosomes as compared to the one or more miRNA control levels. In some embodiments, characterizing the cancer comprises determining a type, a grade, and/or a stage of the cancer. Further, in some embodiments, determining the amount of the one or more miRNAs comprises determining a total amount of the miRNA in the cancer-derived exosomes.

Another embodiment of the second aspect provides a method of screening for cancer in a subject comprising identifying a subject at risk of having cancer or in need of screening wherein an abnormal status of exosomes and/or the exosome-associated marker in the sample indicates the presence of cancer. In some embodiments the subject is at risk for developing a specific cancer type and the abnormal status of exosomes and/or the exosome-associated marker indicates the presence of this specific cancer type.

Another embodiment of the second aspect provides a diagnostic method comprising identifying a subject who is a candidate for biopsy, wherein an abnormal status of exosomes and/or the exosome-associated marker in the sample indicates a biopsy is desirable. In some embodiments no abnormal status of exosomes and/or the exosome-associated marker indicates no biopsy is necessary.

Another embodiment of the second aspect provides a method of detecting a specific disease other than cancer (e.g., rheumatoid arthritis, diabetic nephropathy) comprising determining the status of exosomes and/or an exosome-associated marker in a sample obtained from a subject, wherein an abnormal status of exosomes and/or the exosome-associated marker indicates the subject has the disease.

In a third aspect of the disclosed subject matter, there is provided a method for in vitro screening of a subject for determining the presence of a disease condition in the subject, such method comprising:

a) providing a biological sample obtained from the subject,

b) isolating extracellular vesicles from said sample, wherein this step of isolating extracellular vesicles comprises

1) labeling the extracellular vesicles using fluorescence in situ hybridization, and

2) sorting the labeled extracellular vesicles by high resolution flow cytometry;

c) determining, from the extracellular vesicles isolated in step b), the status of exosomes and/or an exosome-associated marker in the sample obtained from the subject; wherein an abnormal status of exosomes and/or the exosome-associated marker in the sample indicates the presence of a disease condition in the subject.

In some embodiments status is determined by determining the level of an indicator exosome or an exosome-associated marker in the sample, such as by comparing the level of a specific type of exosome, an indicator exosome, with the aggregate level of all exosomes in the sample. This may comprise determining the status (e.g., the level) of an exosome-associated marker. It may also comprise isolating exosomes from the sample and determining the status (e.g., the level) of an exosome-associated marker.

In an embodiment, the level or presence of a suitable biomarker (e.g. a specific miRNA marker or miRNA expression profile is determined in step c), and

d) the status of exosomes and/or an exosome-associated marker in the sample obtained from the subject is determined by comparing the level or presence of the biomarker determined in step c) with one or more reference values.

In another embodiment, the reference value is the level or presence of the same biomarker of step c) in samples obtained from a subject different from than the subject of step a).

In a fourth aspect of the disclosed subject matter, there is provided a method for in vitro screening for the presence of a disease condition in a test subject comprising

a) providing a biological sample obtained from that subject,

b) isolating extracellular vesicles from said sample, wherein this step of isolating extracellular vesicles comprises

1) labeling the extracellular vesicles using fluorescence in situ hybridization, and

2) sorting the labeled extracellular vesicles by high resolution flow cytometry;

c) determining, from the extracellular vesicles isolated in step b), the status of exosomes and/or an exosome-associated marker in a sample obtained from the subject;

d) comparing the status of exosomes and/or an exosome-associated marker in the sample with the status of exosomes and/or the exosome-associated marker to the status of exosomes and/or an exosome-associated marker determined in a population of training subjects known to have the disease condition; wherein a similarity of the status of exosomes and/or the exosome-associated marker of the test subject to the status of exosomes and/or the exosome-associated marker of the training subjects indicates the presence of the disease condition in the subject.

In an embodiment, the level or presence of a suitable biomarker (e.g. a specific miRNA marker or miRNA expression profile associated with the disease condition) is determined in step c), and

d) the status of exosomes and/or an exosome-associated marker in the sample obtained from the subject is determined by comparing the level or presence of the biomarker determined in step c) with one or more reference values of the biomarker determined in the population of the training subjects.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 shows schematically aspects of sorting of EVs by identification of cell-specific EV markers by miRNA sequencing in accordance with one embodiment of the subject matter described herein.

FIG. 2 is a schematic depicting sorting a plurality of heterogeneous EVs by labeling them by origin cell type using MiRNA-FISH and using high-resolution flow cytometry to separate different cell types in accordance with one embodiment of the subject matter described herein.

FIGS. 3A through 3D show photomicrographs of NanoLuc (red) expression iPS cells having the CD63 marker in accordance with one embodiment of the subject matter described herein.

FIG. 4 shows a graph comparing Luminescence of EVs isolated from CD63-nLuc cells and wild-type cells in accordance with one embodiment of the subject matter described herein.

FIG. 5 shows a graph comparing luminescence of cardiac fibroblasts co-cultured with wild type cardiac myocytes or cardiac myocytes expressing CD63-nLuc in accordance with one embodiment of the subject matter described herein.

FIG. 6 shows schematically the application of this methodology to the screening of subjects for Dox cardiomyopathy in accordance with one embodiment of the subject matter described herein.

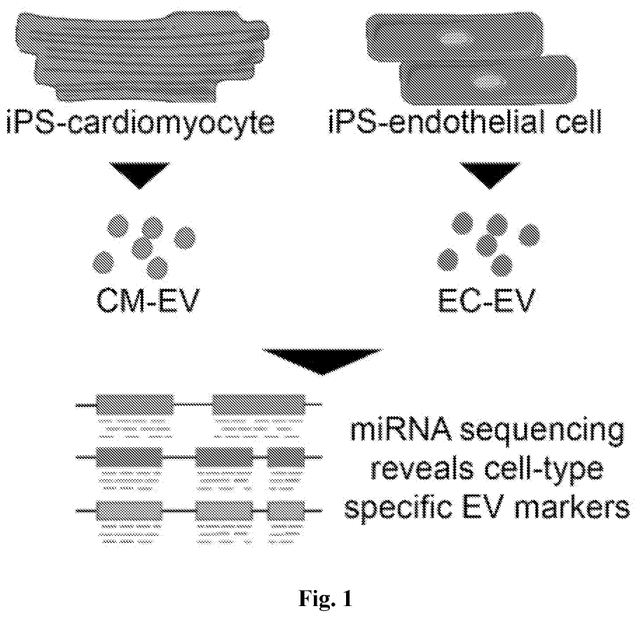

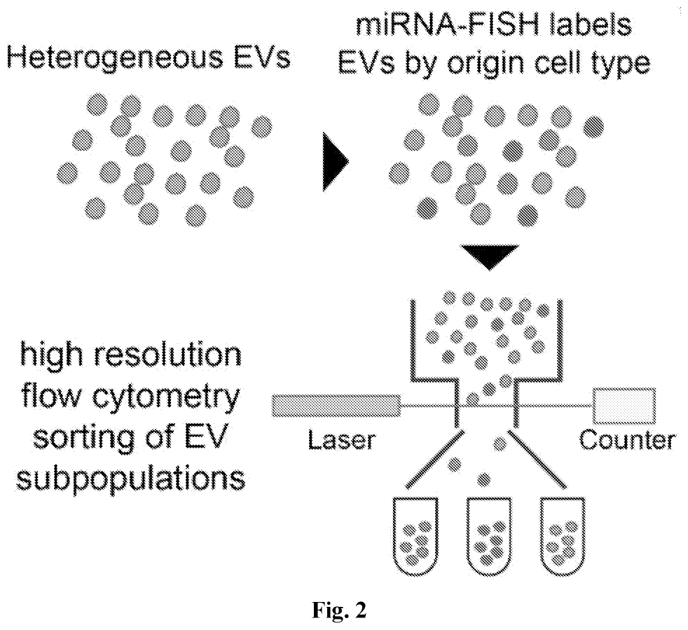

### DETAILED DESCRIPTION OF THE DISCLOSED SUBJECT MATTER

The terms “a”, “an”, and “the” refer to “one or more” when used in this application, including the claims. Thus, for example, reference to “a microRNA” includes a plurality of such microRNAs, and so forth.

The technology described herein improves upon prior diagnostic applications by isolating EVs in a blood sample according to parent cell type. By sorting captured EVs in this manner, this technique facilitates the analysis of the pathophysiological state of the parent cells, rather than the more systemic diagnosis currently possible with EV methods. Since this technology can separate EVs by parent cell type, it may enable concurrent screening of several organ systems from a single subject sample. As such, this technology could lead to unparalleled insight into the pathophysiological status of parent cells and improve the ease of targeted diagnostics for a wide range of diseases.

Extracellular vesicles (EVs) are mobile membrane-limited vesicles secreted from cells into the extracellular environment of multicellular organisms, and can be broadly categorized as microvesicles, apoptotic bodies, and exosomes. They are cell-derived vesicles that circulate in blood and many other bodily fluids and provide a means of intercellular communication. Furthermore, EVs contain a variety of biomaterials relevant to their parent cell, such as RNA, DNA, and a range of proteins and metabolites; therefore, isolation and analysis of EVs from subject blood samples can facilitate disease diagnosis for conditions ranging from cancer to diabetes. This technology proposes a method for separating and analyzing EVs based on their parent cell type. In contrast to current systemic methods, EV-characterization based on parent cells will greatly expand the amount of location- and cell-specific insights possible from this diagnostic method.

Traditional light microscopes generally cannot image EVs, but recent research has relied primarily on flow cytometry and laser confocal microscopy to analyze EVs.

Current characterization methods for EVs include western blot, transmission electron microscopy, or nanoparticle tracking analysis.

EVs have been identified as potentially useful for diagnosing a wide range of diseases, ranging from various types of cancers to heart conditions and other disease conditions.

“Sample” as used herein refers to any biological specimen, including any tissue or fluid, that can be obtained from, or derived from a specimen obtained from, a human or mammalian subject. Such samples include, healthy or tumor tissue, bodily fluids, waste matter (e.g., urine, stool), etc. In some embodiments the sample is blood or any substance derived therefrom—e.g., serum or plasma. Subject blood samples are complex mixtures of circulating elements including, but not limited to, cellular debris (e.g., membrane fragments), free protein, protein aggregates, and small extracellular vesicles (e.g., exosomes). In some embodiments the subject blood sample is enriched for exosomes and/or exosome-associated markers. In other words the sample is enriched to remove cells and individual proteins. The process of extracting plasma from blood, by removing the blood cells, helps to enrich for exosomes and/or exosomes-associated markers. Further purifying plasma to serum, by removing fibrinogen and other clotting factors, further enriches the sample. Additional enrichment/purification may be performed by various techniques including, but not limited to, size exclusion, precipitation, immunoaffinity isolation, ultracentrifugation, filtration, affinity chromatography, antibody capture, cell-sorting, or a combinatory approach. However, these methods for vesicle isolation do not result in the purity levels needed for clinical applications.

Applications of the approach described herein could be useful for diagnosis of solid tumors, therapeutic delivery systems based on modified EVs, evaluation of tumor classification (be it benign, malignant and metastatic) or the transformation status of a tumor (benign to malignant and non-metastatic to metastatic), periodic (e.g., annual) screening of at-risk groups for relevant cancers, monitoring of disease progression for diabetes and other kidney-related conditions, and/or targeted therapeutic delivery for cardiovascular regenerative medicine.

This Technology:

Isolates and separates EVs based on their parent cell type;

Facilitates insight into the pathophysiological state for a given region within the body;

Enables a more personalized analysis of the biomarkers obtained from a given sample of EVs;

Enhances the amount of diagnostic information attainable from a blood sample, while reducing the need for more invasive diagnostic techniques; and

Can improve diagnostic techniques for a range of localized disease states, such as solid tumors or regionalized cardiovascular conditions.

As used herein, the “status” of a biomolecular marker (e.g., exosomes, exosome-associated markers, etc.) refers to the presence, absence, or extent/level of the marker or some physical, chemical, or genetic characteristic of the marker or its expression product(s). Such characteristics include, but are not limited to, expression level, activity level, structure (e.g., sequence information, including mutations), of one or more miRNA moieties. These may be assayed directly (e.g., by assaying the expression level of the miRNA moieties) or determined indirectly (e.g., assaying the level of a miRNA marker whose expression level is correlated to the expression level of the gene of interest).

In some embodiments determining the status of exosomes and/or an exosome-associated marker comprises determining their level in a sample. As used herein in the context of exosomes, markers, etc., the “level” of something in a sample has its conventional meaning in the art. Determining a “level” therefore includes quantitative determinations (e.g., mg/mL, fold change, etc.). Determining a “level” herein also includes more qualitative determinations, e.g., determining the presence or absence of a marker or determining whether the level of the marker is “high,” “low” or even “present” relative to some index value.

“Abnormal status” means a marker's status in a particular sample differs from the reference status for that marker (e.g., in healthy samples or average diseased samples). Examples include mutated (when the sequence or structure of the marker is analyzed), elevated, decreased, present, absent, etc. For example, determining the status of a microscopic RNA marker will often include determining its level in a sample (e.g., on the surface of exosomes). An abnormal status could be either lower (including undetectable) or higher levels (including anything non-zero) compared to the index value in exosome samples from healthy subjects. In this context, for example, a subject has an “increased likelihood of cancer” if the status of the relevant marker in the subject's sample is correlated with cancer. Examples include mutations in particular genes correlated with cancer, a level of the marker that is closer to some cancer index value than to a normal index value, a level of the marker that exceeds some threshold value where exceeding that value is correlated with cancer, etc. Thus “increased likelihood of cancer” means a subject with an abnormal status for a marker has a higher likelihood of cancer than if the subject did not have an abnormal status.

An “elevated status” means that one or more of the above characteristics (e.g., expression) of the marker of interest is higher than normal levels. Generally this means an increase in the characteristic (e.g., expression) as compared to an index value. Conversely a “low status” means that one or more of the above characteristics (e.g., expression) is lower than normal levels. Generally this means a decrease in the characteristic (e.g., expression) as compared to an index value. In this context, a “negative status” generally means the characteristic is absent or undetectable (which would include expression, copy number, methylation, etc.).

In some embodiments, the level of a marker is determined within one or more samples as compared to some index value. Those skilled in the art will appreciate how to obtain and use an index value in the methods of the disclosed subject matter. The index value may represent the average (e.g., mean) level in a plurality of training subjects (e.g., both diseased and healthy subjects). For example, a “cancer index value” can be generated from a plurality of training subjects characterized as having cancer. A “cancer-free index value” can be generated from a plurality of training subjects defined as not having cancer. Thus, a cancer index value may represent the average level of a marker (e.g., exosomes, exosome-associated markers, etc.) in subjects having cancer, whereas a cancer-free index value may represent the average level of the marker in subjects not having cancer. Thus, when the level of the marker is more similar to the cancer index value than to the cancer-free index value, then it can be concluded that the subject has or is likely to have cancer. On the other hand, if the level of the marker is more similar to the cancer-free index value than to the cancer index value, then it can be concluded that the subject does not have, or has no increased likelihood of, cancer.

Alternatively index values may be determined thusly: In order to assign subjects to risk groups, a threshold value may be set for the marker. The optimal threshold value is selected based on the receiver operating characteristic (ROC) curve, which plots sensitivity vs. (1 minus specificity). For each increment of the marker mean (e.g., exosome-associated marker expression), the sensitivity and specificity of the test is calculated using that value as a threshold. The actual threshold will be the value that optimizes these metrics according to the artisan's requirements (e.g., what degree of sensitivity or specificity is desired, etc.).

Determining the status of exosomes in a sample may also comprise assaying some marker whose status itself is correlated with exosome status. This marker will often be an exosome-associated marker. “Exosome-associated” marker means a biomolecule inside or on the surface of an exosome. For example, exosomes carry cellular biomolecules such as proteins and nucleic acids (e.g., mRNA, microRNA, etc.) within their lipid membrane. Since exosomes are formed from membranes of the cell, they also carry molecules (e.g., cell-surface molecules such as CD63 or EpCAM) on their surface. Additionally, since some biomolecules bind those on the surface of exosomes (e.g., antibodies binding an exosome-surface protein), these biomolecules are also indirectly “exosome-associated.” As discussed below, in some embodiments the methods described need not be limited by the actual physical element that is measured in the claimed method as long as the status of an exosome-associated marker is determined. Thus, the invention envisions detecting an exosome-associated marker even after such marker has been separated from the exosome with which it is sometimes associated.

Expression profiling technologies have identified new biomarkers with diagnostic applications. One such biomarker group is a class of small non-coding RNAs, termed microRNAs (miRNAs). MicroRNAs are small (22-25 nucleotides in length non-coding RNAs that suppress the translation of target mRNAs by binding to their 3′ untranslated region. Post-transcriptional silencing of target genes by miRNA can occur either by cleavage of homologous mRNA or by specific inhibition of protein synthesis.

As discussed further below, analysis of exosomal miRNA can be used to identify the type and health status of the parent cell of the exosome. The presently disclosed subject matter provides for the determination of the amount of exosomal miRNAs correlated with a disease condition within biological fluids of a subject, and in particular, from serological samples from a subject, such as for example blood. This provides the advantage of biological samples for testing that are easily acquired from the subject. The amount of one or more miRNAs of interest in the biologic sample can then be determined as described herein and compared to miRNA control levels.

The “amount” of one or more miRNAs determined refers to a qualitative (e.g., present or not in the measured sample) and/or quantitative (e.g., how much is present) measurement of the one or more miRNAs. The “control level” is an amount (including the qualitative presence or absence) or range of amounts of one or more miRNAs found in a comparable biological sample in subjects not suffering from the disease condition. As one non-limiting example of calculating the control level, the amount of one or more miRNAs of interest present in a normal biological sample (e.g., blood) can be calculated and extrapolated for whole subjects.

A disease condition of interest comprises cancer.

Tumors analyzed by miRNA profiling have exhibited significantly distinct miRNA signatures, compared with normal cells from the same tissue. An analysis of leukemias and solid cancers determined that miRNA-expression profiles could classify human cancers by developmental lineage and differentiation state. The expressions of individual miRNAs and specific miRNA signatures can be linked to the diagnosis and prognosis of many human cancers.

As used herein, “cancer type” means a cancer in or originating from a particular tissue or organ and/or a cancer with a particular molecular or clinical feature. Often, the specificity of the “cancer type” varies with the application, including tissue type (e.g., squamous versus cuboidal), organ type (e.g., breast versus lung), and clinical subtype (e.g., triple-negative breast cancer).

Some microRNAs that may be used as indicators for cancer in a subject include one or more microRNA selected from the group consisting of miR-016, miR-019b, miR-020a, miR-21, miR-024, miR-026a, miR-32, miR-126, miR-141, miR-146a, miR-146b, miR-191, miR-200a, miR-200b, miR-200c, miR-203, miR-205, miR-214, miR-222, miR-223, and miR-484.

### Identifying Novel Cell-type Specific EV Markers

Provided is a method for identifying cell-type specific EV markers, the method comprising

culturing induced pluripotent stem cells (iPSCs) to provide a pure population of a first differentiated cell type therefrom;

isolating extracellular vesicles (EV)s from the culture of the differentiated cell type;

generating a microscopic RNA (miRNA) expression profile for the differentiated cell type by next-generation sequencing; and

identifying specific miRNA markers associated with the pure population of the differentiated cell type by differential expression analysis by comparison to a miRNA expression profile of a population of non-differentiated iPSCs and/or a second differentiated cell type different from the first differentiated cell type.

For example but not limitation, the method is described specifically for EVs from cells of the cardiovascular system as a model organ, establishing methods that can be broadly applied to EVs from all other organ systems.

In a first example embodiment of this method, the miRNA expression profile of EVs from a culture of pure iPS-cardiomyocytes provides a miRNA profile associated with a healthy or normal cardiomyocyte, which can be compared to a sample of EVs obtained from a culture of non-differentiated IPSCs. The differences between the miRNA expression profiles can be analyzed to identify miRNA biomarkers associated with a differentiated cardiomyocyte. In some embodiments, the quantitated expression of a specific miRNA may be used to define a threshold level for that miRNa that is characteristic for that cell type.

EVs were isolated from both induced pluripotent stem cells (iPSCs) and cardiomyocytes differentiated from iPSCs (iPS-CMs). Using next generation miRNA sequencing, we discovered that EVs isolated from iPS-CMs carry a unique set of miRNAs when compared to EVs isolated from iPSCs. Specifically, I have found that iPS-CM EVs carry muscle specific miRNAs including miR-1 and miR-133a. This work can be expanded to identify novel cell-type specific markers for EVs secreted by other cell types important in cardiovascular pathophysiology.

Using the same methods, iPSCs can be used to differentiate all relevant cell types including atrial cardiomyocytes, ventricular cardiomyocytes, cardiac fibroblasts, macrophages, and endothelial cells. These differentiation protocols have been previously described to yield highly pure populations of resulting cells lending to their utility in generating pure cell-type specific EVs. Isolation of EVs from each of these different cell types separately provides a “normal” population of EVs for each cell type. Use of next-generation sequencing generates a miRNA expression profile for EVs from each cell type. Differential expression analysis of the resulting cell-type specific profiles will allow for identifying specific miRNA markers that define each EV by their parent cell type.

In another example embodiment, the miRNA expression profile of EVs from a culture of pure iPS-cardiomyocytes prepared as described above provides a miRNA profile associated with a healthy or normal cardiomyocyte, which can be compared to a sample of EVs obtained from a subject known to be suffering from a cardiovascular disease condition to provide a differential miRNA profile, wherein the absence, underexpression, presence and/or overexpression of a specific miRNA associated with the cell type (such as determined in the first example embodiment) provides a biological marker associated with the disease condition. For example, a miRNA present in the EVs associated with cells having a known disease condition that is not present in the miRNA expression in a healthy cell may be indicative of the disease condition. Conversely, a miRNA absent in the EVs associated with cells having a known disease condition that is present in the miRNA expression in a healthy cell may also be indicative of the disease condition.

Embodiments of this aspect of the disclosed subject matter include those wherein the induced pluripotent stem cells (iPSCs) are organ specific.

In a particular embodiment, the iPSCs are specific to the cardiovascular system.

In particular embodiments, the iPSCs are cardiomyocytes, atrial cardiomyocytes, ventricular cardiomyocytes, cardiac fibroblasts, macrophages or endothelial cells.

In other embodiments, the iPSCs are specific to one of the following organs: skin; lung; colon; breast; prostate; ovaries; endometrium; uterus; cervix; testes; esophagus; stomach; kidney; liver; bladder; urinary tract; pancreas; brain; thyroid; or central nervous system.

Notably the method described above can be used to characterize a miRNA marker associated with a cancerous condition. In embodiments, the method can be used to identify one or more miRNA markers associated with one of the following cancer cell types: benign tumor; malignant tumor; metastatic tumor; carcinoma; sarcoma; melanoma; lymphoma; leukemia; myeloma; squamous tumor; cuboidal tumor; glioblastoma; malignant peripheral nerve sheath tumor (MPNST); triple-negative breast cancer; or HER2-overexpressing breast cancer.

The miRNA markers discovered by the method described herein can be used to identify a cancer type associated with a specific organ, allowing for diagnosis of a cancerous condition by non-invasive means. Further, when a miRNA marker is identified for a cancerous condition specific to a particular organ and/or cancer type, it can be used to screen a population likely to be susceptible to such cancer to provide early diagnosis of a possible cancerous condition, in some instances before other symptoms associated with the cancer can be observed.

In addition to identifying and screening for MiRNA markers related to cancerous conditions, the methods described herein are also useful for characterizing other disease conditions such as chronic or acute inflammatory diseases, such as wherein the inflammation is associated with an infectious disease, an autoimmune disease, hypersensitivity-associated inflammation, graft rejection, injury, disorders of apoptosis or diseases associated with activation of the necroptosis activation pathway.

Inflammatory Disease. Inflammation is a process whereby the immune system responds to infection or tissue damage. Inflammatory disease results from an activation of the immune system that causes illness, in the absence of infection or tissue damage, or at a response level that causes illness. Inflammatory disease includes autoimmune disease, which includes any disease caused by immunity that becomes misdirected at healthy cells and/or tissues of the body. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

Inflammatory diseases of interest include, without limitation, graft versus host disease, Secondary Progressive Multiple Sclerosis (SPMS); Primary Progressive Multiple Sclerosis (PPMS); Neuromyelitis Optica (NMO); Psoriasis; Psoriatic Arthritis; Systemic Lupus Erythematosis (SLE); Ulcerative Colitis; Crohn's Disease; Ankylosing Spondylitis; Rheumatoid Arthritis (RA); Diabetes Mellitus type 1 (IDDM); Asthma; Chronic Obstructive Pulmonary Disorder (COPD); Chronic Hepatitis; Amyotrophic Lateral Sclerosis (ALS); Alzheimer's Disease (AD); Parkinson's Disease; Frontotemporal Lobar Degeneration (FTLD), atherosclerosis/cardiovascular disease, and obesity/metabolic syndrome. Applying the methods of the invention with respect to identifying mechanistic biomarkers to these other diseases leads to identification of biomarkers suitable for a diagnostic to predict response to therapy.

The term “necroptosis” as used herein refers to a programmed necrotic cell death. Necroptosis is also referred to as necrosis associated with inflammation. Characteristically, necroptosis involves cellular swelling and rupture, thereby releasing the intracellular contents.

The phrase “disease associated with activation of a necroptosis activation pathway” as used herein refers to any disease or disorder which involves activation of a component of the necroptosis activation pathway. Exemplary diseases include, but are not limited to, necroptosis, inflammation, necroptosis associated with inflammation, necroptosis associated with an infection, tissue damage, tissue injury, myocardinal infarction (MI), stroke, ischemia-reperfusion injury (IRI), atherosclerosis, psoriasis, psoriatic arthritis, rheumatoid diseases (e.g. Rheumatoid arthritis), pancreatitis, diabetes, asthma, emphysema, kidney tissue damage (e.g. Acute tubular necrosis), autoimmune disease (e.g. multiple sclerosis, lupus), inflammatory bowel disease (IBD), Ulcerative colitis (UC), Crohn's disease (CD), neurodegeneration (e.g. Parkinson's disease, Alzheimer's disease), and graft related diseases (e.g. graft rejection and graft versus host disease). According to a specific embodiment the disease is a necroptosis, an inflammation, a necroptosis associated with inflammation, a necroptosis associated with an infection, a brain tissue damage or injury (e.g. neurodegeneration or stroke), a kidney tissue damage or injury (e.g. Acute tubular necrosis), a lung tissue damage or injury (e.g. emphysema), a cardiac tissue damage or injury (e.g. MI) or a gastrointestinal tissue damage or injury (e.g. IBD, UC or CD).

In some embodiments, the miRNA markers are specific to one of the following disease conditions: myopathy; cardiomyopathy; neuropathy; neuromyopathy; diabetic nephropathy; myoclonus; diabetes; cardiovascular disease; Parkinson's disease; Alzheimer' s disease; dystonia musculorum deformans; radiation exposure; graft vs. host disease; apoptosis; necroptosis; or adverse pregnancy risk.

The disclosures herein are useful for a method for determining in vitro the presence of a disease condition in a subject, such method comprising:

a) providing a biological sample obtained from that subject,

b) isolating extracellular vesicles from said sample, wherein this step of isolating extracellular vesicles comprises

1) labeling the extracellular vesicles using fluorescence in situ hybridization, and

2) sorting the labeled extracellular vesicles by high resolution flow cytometry;

c) determining, from the extracellular vesicles isolated in step b), the status of exosomes and/or an exosome-associated marker in a sample obtained from the subject; wherein an abnormal status of exosomes and/or the exosome-associated marker in the sample indicates the presence of a disease condition in the subject.

In an embodiment, at least a portion of the extracellular vesicles are exosomes.

In a further embodiment, the extracellular vesicles are exosomes.

In one embodiment the subject is a human.

In another embodiment the subject is a mammal.

In one embodiment, the test is an in vitro test.

In another embodiment, the biomarker of step c) is miR-1.

In another embodiment, the biomarker of step c) is miR-133a.

In another embodiment, the biomarker of step c) is one or more microRNA selected from the group consisting of miR-016, miR-019b, miR-020a, miR-21, miR-024, miR-026a, miR-32, miR-126, miR-141, miR-146a, miR-146b, miR-191, miR-200a, miR-200b, miR-200c, miR-203, miR-205, miR-214, miR-222, miR-223, and miR-484.

In another embodiment, the biomarker of step c) is RNU6.

In one embodiment, the biological sample of step a) is obtained from a subject affected by a benign tumor.

In a particular embodiment, the benign tumor is a benign colon tumor.

In a particular embodiment, the benign tumor is a plexiform neurofibroma.

In one embodiment, the sample is a tumor sample.

In another embodiment, the sample is a bodily fluid.

In a particular embodiment, the sample is a plasma sample.

In a particular embodiment the sample is a blood sample.

In a particular embodiment the sample is a serum sample.

In a particular embodiment the sample is a urine sample.

In a particular embodiment the sample is a saliva sample.

In one embodiment the tumor transformation status is the transformation to an MPNST.

In another embodiment, the tumor transformation status is the transformation to a colorectal cancer.

In one embodiment the subject is suspected of being affected by a tumor.

In one embodiment, the tumor is a malignant tumor.

In one embodiment, the tumor is colon cancer.

In another embodiment, the tumor is gastric cancer.

In another embodiment, the tumor is breast cancer.

In another embodiment, the tumor is lung cancer.

In another embodiment, the tumor is melanoma.

In another embodiment, the tumor is pancreatic cancer.

In another embodiment, the tumor is ovarian cancer.

In another embodiment, the tumor is prostate cancer.

In another embodiment the tumor is a central nervous system tumor.

In a particular embodiment, the central nervous system tumor is glioblastoma.

In another embodiment, the tumor is MPNST.

Any combination of the above embodiments of this second aspect of the invention represent further embodiments of the invention.

### Sorting of EV Subpopulations By High Resolution Flow Cytometry

The sorting of EVs based on their parent cell type and the subsequent analysis of these subpopulations will allow for increased insight into the physiological status of the tissue of origin. Emerging technologies including miRNA fluorescent in-situ hybridization (FISH), high resolution flow cytometry, and FISH-based flow cytometry now enable the ability to sort EVs based on miRNA content.

For both fundamental and clinical studies the development of techniques that enable individual EV-based high throughput multi-parameter analysis and isolation is of great importance to investigate changes in (minor) EV subsets and to unravel (functional) differences. Since the vast majority of EVs are less than 200 nm in size, this is a major challenge in the EV field. Flow cytometry is a technique that is designed for high-throughput, multi-parameter analysis of particles in suspension and has the ability to sort out specific subsets. However, typical flow cytometry cannot easily sort particles of that size.

Particle concentrations in EV samples are often unknown and hard to predict. At high concentrations however, coincidence can occur during flow cytometric measurement of submicron-sized particles. Coincidence in flow cytometry occurs when two or more particles arrive at the measuring spot at the same time. As a result, the signals derived from multiple particles are added up and detected by the flow cytometer as a single event. As the number of coincidences further increases due to increasing event rates, this will ultimately result in the presence of a permanent scatter and fluorescent signal in the measuring spot. Consequently, separate events can no longer be distinguished, event rates will drop, and scatter and fluorescent signals will be greatly overestimated. This special form of coincidence is also referred to as “swarm”.

Most current flow cytometry systems are designed to run with a sheath fluid surrounding the core sample stream, with a core diameter and laser spot size well suited to accommodate particles in the size range of cells. However, when measuring submicron-particles such as EVs, the spot size in relation to EV concentration should be adjusted to avoid unwanted coincidence effects.

High resolution flow cytometry is well-suited to the methods of the invention since multiple markers may be assayed at once by analyzing a panel of antigens by cell sorting using antibodies to each antigen and counting on a multichannel sorter. High resolution flow cytometry can be based on fluorescence threshold triggering. This implies that signal detection and sample quantification are dependent on fluorescence intensity and independent of size and refractive index (RI), allowing for the detection of EVs ranging from 100-200 nm equivalent fluorescence.

Thus the disclosures herein include a method of quantitating exosomes comprising isolating exosomes from a subject sample and counting the exosomes using high resolution flow cytometry. In some embodiments the isolation and counting may be done simultaneously, such as by using miRNA fluorescent in-situ hybridization (FISH) adapted for use with exosomes. FISH can be used to fluorescently label the miRNA within EVs and allow for them to be sorted using high resolution flow cytometry.

Multiplexed miRNA fluorescence in situ hybridization (miRNA FISH) is an advanced method for visualizing differentially expressed miRNAs. As discussed above, some miRNAs are excellent disease biomarkers due to their abundance and cell-type specificity. However, these short RNA molecules are difficult to visualize due to loss by diffusion, probe mishybridization, and signal detection and signal amplification issues. A reliable and adjustable method for visualizing and counting EVs from a specific cell type was developed, based on the EV membrane marker CD63 linked to NanoLuc luciferase. CD63 linked to NanoLuc luciferase is a protein-coupled marker. Alternatively, miRNAs can be coupled to fluorescent markers providing a miRNA-coupled fluorescent marker that can be used to fluorescently label EVs.

The fluorescently-labeled EVs can be sorted and counted using high-resolution flow cytometry.

High-resolution flow cytometry of fluorescently labeled extracellular vesicles can be performed on a jet-in-air-based BD Influx flow cytometer (BD Biosciences) using an optimized configuration. In brief, the BD Influx is triggered on the fluorescence signal derived from the fluorescently labeled particles (FL1 signal) and thresholding was applied on this fluorescence channel. The maximum PMT voltage may be 1250 V and the trigger signal PMT may be used at 42%. The threshold level can be adjusted to allow an event rate of less than or equal to about 10 events per second when running clean PBS.

For sorting experiments, the system and procedure are optimized to allow for maximal event rates and small sort volumes, while avoiding coincidence. Therefore, a 70 μm nozzle may be used and sheath fluid pressure may be increased to 30 psi.

The sample fluid pressure is increased accordingly (such as to 29 psi) to reach an event rate of less than 5,000 per second (higher event rates can result in coincidence and swarm). With a drop frequency of 49.22 kHz, sort efficiencies (greater than 95%) can be maintained at acceptable levels.

Using the techniques described above, we have discovered miR-1 as a potential marker separating cardiomyocyte EVs from EVs secreted by other cells in the heart. To specifically isolate the population of miR-1 expressing EVs, a method to sort EVs using high resolution flow cytometry can be based on fluorescently labeled miRNAs. Fluorescently linked miR-1 probes can be used to perform miRNA FISH on EVs isolated from iPS-CMs. To assay for the specificity of sorting, EVs from iPSCs which do not express miR-1 but express the iPSC specific miR-302 can be labelled with miR-302 probes and mixed with iPS-CM EVs. miRNA expression profiling can be conducted on the two sorted subpopulations of EVs in order to confirm accuracy of the sort. Combining this methodology with the EV markers identified using the methods described above will allow for establishing a set of miRNA probes that define the origin cell type of each EV and accurately sort them into cell-type specific subpopulations.

A rodent animal model can be used to validate and optimize this method for the isolation of EV subpopulations from blood plasma. An EV mixture containing EVs from different iPSC-derived cardiac cell types can be introduced intravenously into Sprague-Dawley rats. Plasma is extracted from animals and all systemic plasma EVs are isolated. Systemic plasma EVs are sorted into cell-type specific subpopulations based on their expression of specific miRNA markers. Each cell-type specific subpopulation then undergoes miRNA expression profiling to confirm the fidelity of EV sorting.

In a specific example, we have developed novel methods for the isolation and separation of extracellular vesicles (EVs) based on their parent cell-type in order to advance the utility of EVs as potent biomarkers for health and disease. To characterize differences in EVs from multiple cell types, we have established an induced pluripotent stem cell (iPSC) line that stably expresses the EV membrane marker CD63 linked to NanoLuc luciferase, allowing for efficient and robust tracking of all secreted EVs. Leveraging the ability of iPSCs to differentiate into multiple cell types, we have demonstrated that mesenchymal stem cells, cardiac fibroblasts, and cardiac myocytes derived from CD63-nLuc iPSCs stably express the NanoLuc-linked CD63.

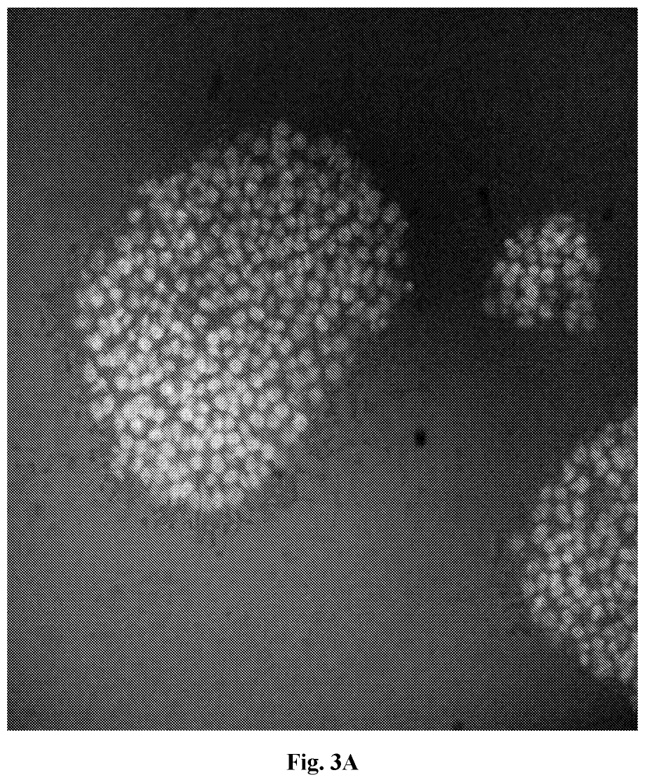

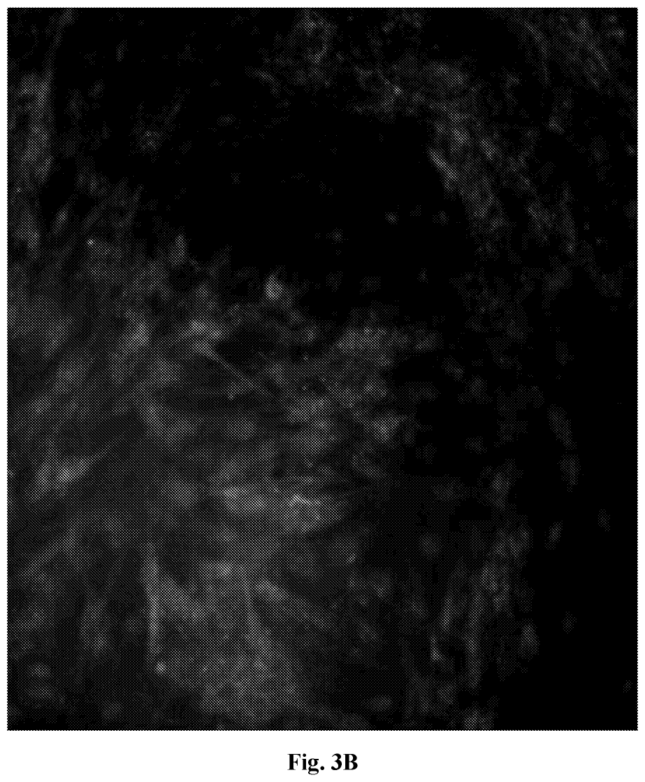

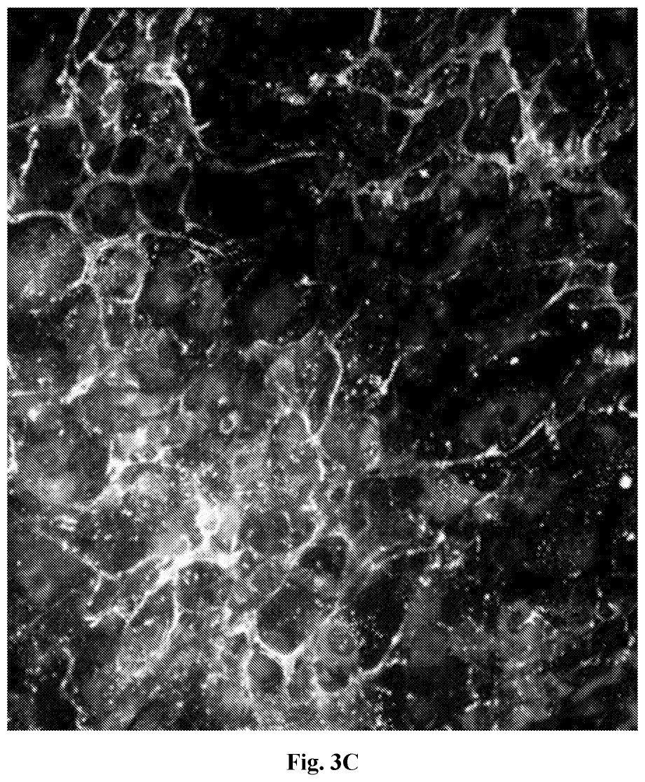

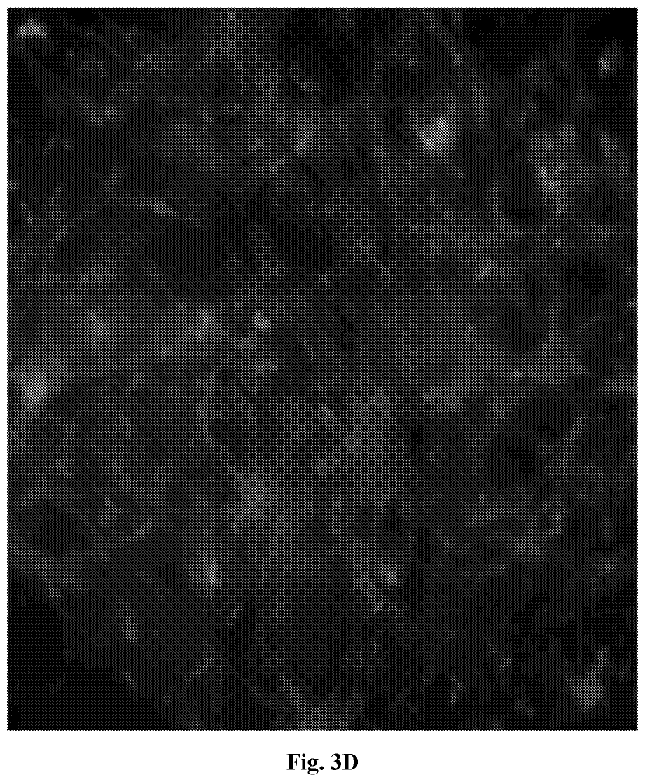

Photomicrographs of NanoLuc (red) expression in iPSCs having the CD63 marker are shown in FIGS. 3A-3D. FIG. 3A shows a photomicrograph of iPSCs stained with Nanog (teal-generally brighter circles). FIG. 3B shows a photomicrograph of mesenchymal stem cells stained with CD90 (magenta). FIG. 3C shows a photomicrograph of cardiac fibroblasts stained with Collagen I (white). FIG. 3D shows a photomicrograph of cardiac myocytes stained with cardiac troponin (blue).

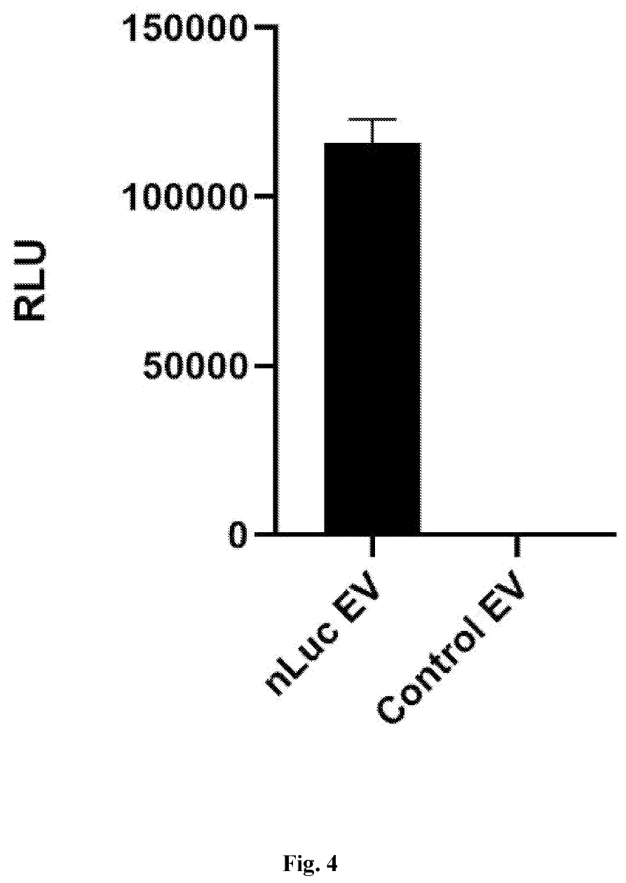

We next demonstrated luminescence of EVs secreted by CD63-nLUC expressing cells. EVs were isolated from cell-conditioned supernatant using ultracentrifugation and subsequently assayed for NanoLuc luciferase activity demonstrating strong luciferase expression when compared with EVs isolated from control cell-conditioned supernatant (FIG. 4).

FIG. 4 shows a graph comparing Luminescence of EVs isolated from CD63-nLuc cells and wild-type cells.

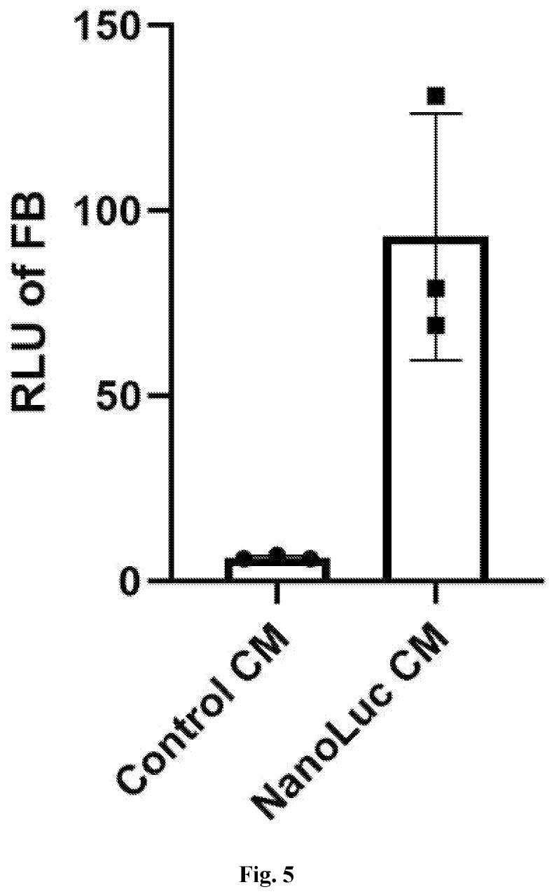

To determine the bioactivity of CD63-nLUC EVs and to assess our ability to track CD63-nLUC EVs across different cell types, we co-cultured wild-type cardiac fibroblasts with either CD63-nLuc cardiac myocytes or wild-type cardiac myocytes in a transwell system. The transwell system allows for the exchange of extracellular vesicles but not direct contact between cardiac fibroblasts and cardiac myocytes. Subsequent analysis of luciferase activity of cardiac fibroblasts demonstrated that fibroblasts co-cultured with nLuc-CMs had a significant increase in luminescence when compared to those cultured with control CMs (FIG. 5).

FIG. 5 shows a graph comparing luminescence of cardiac fibroblasts co-cultured with wild type cardiac myocytes (control CM) or cardiac myocytes expressing CD63-nLuc (NanoLuc CM).

Together, these results demonstrate that the iPSCs expressing CD63-nLUC can be differentiated into many cell-types and that EVs isolated from each of these cell-types can be tracked robustly through their expression of NanoLuc luciferase.

The development of this technology enables accurate tracking of EVs isolated from different cell-types in order to identify cell-type specific cargo as well as the cell-type specific bio-active properties of these EVs.

In the fourth aspect of the disclosed subject matter, there is provided a method for in vitro screening for the presence of a disease condition in a test subject comprising

a) providing a biological sample obtained from that subject,

b) isolating extracellular vesicles from said sample, wherein this step of isolating extracellular vesicles comprises

1) labeling the extracellular vesicles using fluorescence in situ hybridization, and

2) sorting the labeled extracellular vesicles by high resolution flow cytometry;

c) determining, from the extracellular vesicles isolated in step b), the status of exosomes and/or an exosome-associated marker in a sample obtained from the subject;

d) comparing the status of exosomes and/or an exosome-associated marker in the sample with the status of exosomes and/or the exosome-associated marker to the status of exosomes and/or an exosome-associated marker determined in a population of training subjects known to have the disease condition; wherein a similarity of the status of exosomes and/or the exosome-associated marker of the test subject to the status of exosomes and/or the exosome-associated marker of the training subjects indicates the presence of the disease condition in the subject.

A specific example of this fourth aspect is screening for doxorubicin cardiotoxicity.

Doxorubicin (Dox) is a highly effective drug for treating human cancers. Its mechanism of action is by intercalation into DNA and inhibition of topoisomerase 2 that shuts down DNA replication in rapidly dividing tumor cells. Subjects undergoing chemotherapy have increased risk of developing cardiovascular complications that evolve to the heart failure in susceptible individuals.

Current biomarkers for Dox toxicity rely on evidence of cardiomyocyte necrosis (troponin and LDH) and vascular congestion from ventricular dysfunction (BNP), all of which develop after the cellular and clinical toxicity are already present. The use of cell-type specific EVs as a biomarker could predict a subject's risk of developing Dox cardiomyopathy and would have significant clinical utility, allowing development of mitigating therapies, alternative regimens and closer clinical follow ups.

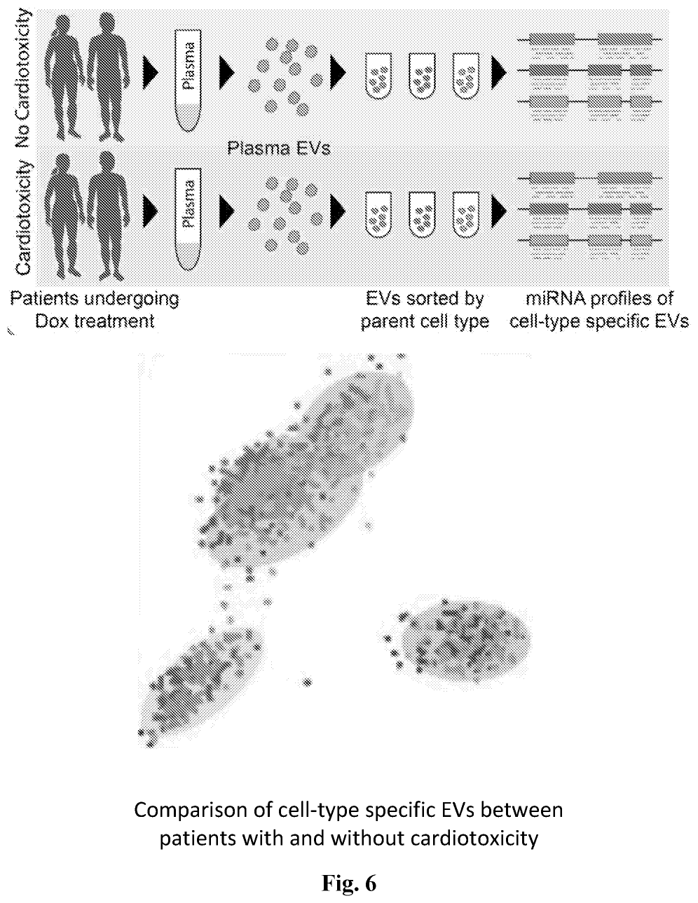

FIG. 6 shows schematically the application of this methodology to the screening of subjects for Dox cardiomyopathy. Blood is collected from subjects undergoing Dox treatment to assess cell-type specific EV miRNA profiles. A shown schematically in FIG. 6, patients (subjects) undergoing Dox treatment are divided into two training populations, the first comprising patients exhibiting no cardiotoxicity and the second comprising patients exhibiting cardiotoxicity. Notably, both populations comprise patients having cancer that are undergoing Dox chemotherapy. Such subjects may have different EV profiles from subjects without cancer and/or who are not being treated with Dox chemotherapy. Optionally, a third training population (not shown in FIG. 6) may be included in the development of the screening method, comprising subjects without cancer to provide an EV or exosome expression profile of a “normal” population to facilitate identification of biomarkers associated with the cancer and/or associated with cardiotoxicity.

Because doxorubicin-mediated cardiotoxicity can occur during treatment or many years after, it is preferable that patients for the first training population not exhibiting cardiotoxicity are selected early in their Dox chemotherapy and patients in the second training population exhibiting cardiotoxicity are selected late in or after their Dox chemotherapy.

All systemic EVs from subject plasma samples in each training population will be collected. The EVs will then be sorted by their parent cell types using the methods detailed above. Comparison of cell-type specific EVs between patients with and without cardiotoxicity will be conducted. Each subpopulation will be characterized using miRNA sequencing. A difference in miRNA expression between the two training groups, or among the three training groups, will determine a miRNA biomarker useful for identifying cardiotoxicity. It may be desirable to develop machine-learning algorithms to synthesize the collected data. By comparing samples from Dox-susceptible subjects after development of cardiotoxicity to samples from subjects who never develop cardiotoxicity, these algorithms will be able to determine biomarker(s) that can predict a test subject's risk of developing Dox-mediated cardiotoxicity before clinical signs become apparent. These biomarkers could include the presence or absence of one or more specific miRNAs or a threshold level for one or more specific miRNAs indicative of Dox-mediated cardiotoxicity.

Once identified, the biomarker(s) can be used in screening new subject(s) for Dox-mediated cardiotoxicity.

After identification of the biomarker(s), screening of test subjects can begin using the method for screening for a disease condition according to the second aspect of the disclosed subject matter. In this example, the disease condition comprises Dox-mediated cardiotoxicity.

In an embodiment, the test subject is a patient having cancer that may be treated using doxorubicin and the test subject is screened prior to initiation of Dox chemotherapy to look for biomarkers indicative of Dox-mediated cardiotoxicity. If a biomarker indicative of Dox-mediated cardiotoxicity is determined in the test subject, Dox chemotherapy may be contraindicated, and/or protocols comprising mitigating therapies, alternative regimens and/or closer clinical follow ups may be initiated prior to treatment of the test subject.

For ongoing screening of a test subject undergoing Dox chemotherapy, samples will be collected regularly (e.g. every month) during Dox treatment and every 6 months for 5 years after the secession of Dox treatment to continue to look for biomarker(s) indicative of cardiotoxicity. If such biomarkers are determined in the test subject, Dox chemotherapy may be terminated, and/or protocols comprising mitigating therapies, alternative regimens and/or closer clinical follow ups may be initiated.

EVs can accurately convey complex information about the pathophysiological status of their parent cells. Here, I establish methodology that enable the sorting of EVs from subject plasma based on their parent cell type. I focused on identifying and sorting EVs from cells of the cardiovascular system in order to develop a predictive model for doxorubicin mediated cardiotoxicity. However, the methods established in this disclosure can be broadly applied to the development of sophisticated EV biomarkers across organ systems.

Similar to how single cell RNA sequencing vastly improved the power of transcription profiling, the ability to sort EVs based on cell type will greatly increase the utility of EVs as a biomarker capable of providing unparalleled insight into the complex and dynamic behaviors of cells, tissues, and organ systems.

1. A method for identifying cell-type specific EV markers, the method comprising
culturing induced pluripotent stem cells (iPSCs) to provide a culture comprising a pure population of a first differentiated cell type therefrom;
isolating extracellular vesicles (EV)s from the culture of the differentiated cell type;
generating a microscopic RNA (miRNA) expression profile for the differentiated cell type by next-generation sequencing; and
identifying specific miRNA markers associated with the pure population of the differentiated cell type by differential expression analysis by comparison to a miRNA expression profile of a population of a second differentiated cell type different from the first differentiated cell type.

2. The method of claim 1 wherein the induced pluripotent stem cells (iPSCs) are hybridized with an EV membrane marker CD63 linked to NanoLuc luciferase.

3. The method of claim 2 wherein the first differentiated cells retain the EV membrane marker CD63 linked to NanoLuc luciferase from their parent (iPSCs) hybridized with an EV membrane marker CD63 linked to NanoLuc luciferase.

4. The method of claim 2 the second differentiated cells are labeled with the EV membrane marker CD63 linked to NanoLuc luciferase.

5. The method of claim 1 wherein isolating extracellular vesicles (EV)s from the culture of the differentiated cell type comprises high resolution flow cytometry.

6. The method of claim 1 wherein the induced pluripotent stem cells (iPSCs) are organ specific.

7. The method of claim 6 wherein the iPSCs are specific to the cardiovascular system.

8. The method of claim 7 wherein the first differentiated cells comprise cardiomyocytes, atrial cardiomyocytes, ventricular cardiomyocytes, cardiac fibroblasts, macrophages or endothelial cells.

9. A method for determining in vitro the presence of a disease condition in a subject, such method comprising:
a) providing a biological sample obtained from the subject,
b) isolating extracellular vesicles from said sample, wherein this step of isolating extracellular vesicles comprises
1) labeling the extracellular vesicles using fluorescence in situ hybridization, and
2) sorting the labeled extracellular vesicles by high resolution flow cytometry;
c) determining, from the extracellular vesicles isolated in step b), the status of exosomes and/or an exosome-associated marker in a sample obtained from the subject;
wherein an abnormal status of exosomes and/or the exosome-associated marker in the sample indicates the presence of a disease condition in the subject.

10. The method of claim 9 wherein sorting the labeled extracellular vesicles includes sorting them by parent cell type.

11. The method of claim 9 wherein the exosome or the exosome-associated marker in the sample is characterized by a specific miRNA marker.

12. The method of claim 9 wherein status is determined by determining the level of an indicator exosome or an exosome-associated marker in the sample.

13. The method of claim 12 wherein status is determined by comparing the level of the indicator exosome or an exosome-associated marker with the aggregate level of all exosomes in the sample.

14. The method of claim 9 wherein the level or presence of a suitable biomarker is determined in step c), and
d) the status of exosomes and/or an exosome-associated marker in the sample obtained from the subject is determined by comparing the level or presence of the biomarker determined in step c) with one or more reference values.

15. The method of claim 14 wherein the suitable biomarker is a specific miRNA marker or miRNA expression profile.

16. The method of claim 14 wherein the reference value is the level or presence of the same biomarker of step c) in an earlier sample from the same subject as in step a).

17. The method of claim 14 wherein the reference value is the level or presence of the same biomarker of step c) in samples obtained from a subject different from than the subject of step a).

18. The method of claim 9 wherein the method is used for determining in vitro the tumor transformation status in a subject.

19. The method of claim 9 wherein the method is used for screening for cancer in a subject, comprising identifying a subject at risk of having cancer or in need of screening wherein an abnormal status of exosomes and/or the exosome-associated marker in the sample indicates the presence of cancer.

20. The method of claim 19 wherein the subject is at risk for developing a specific cancer type and the abnormal status of exosomes and/or the exosome-associated marker indicates the presence of this specific cancer type.

21. The method of claim 9 wherein the method is used for detecting recurrence of cancer in a subject, wherein an abnormal status of exosomes and/or the exosome-associated marker in the sample indicates recurrence.

22. The method of claim 9 wherein the method is used for providing a diagnostic method comprising identifying a subject who is a candidate for biopsy, wherein an abnormal status of exosomes and/or the exosome-associated marker in the sample indicates a biopsy is desirable.

23. The method of claim 9 wherein no abnormal status of exosomes and/or the exosome-associated marker indicates no biopsy is necessary.

24. The method of claim 9 wherein the exosome-associated marker is a cancer-marker, a cancer-type marker, or a tissue-type marker.

25. The method of claim 9 wherein the method is used for detecting a specific disease other than cancer comprising determining the status of exosomes and/or an exosome-associated marker in a sample obtained from a subject, wherein an abnormal status of exosomes and/or the exosome-associated marker indicates the subject has the disease.

26. The method of claim 25 wherein the exosome-associated marker is a marker indicative of inflammatory disease, infectious disease, an autoimmune disease, hypersensitivity-associated inflammation, graft rejection, injury, disorder of apoptosis or disease associated with activation of the necroptosis activation pathway.

27. A method for in vitro screening of a subject for determining the presence of a disease condition in the subject, such method comprising:
a) providing a biological sample obtained from the subject,
b) isolating extracellular vesicles from said sample, wherein this step of isolating extracellular vesicles comprises
1) labeling the extracellular vesicles using fluorescence in situ hybridization, and
2) sorting the labeled extracellular vesicles by high resolution flow cytometry;
c) determining, from the extracellular vesicles isolated in step b), the status of exosomes and/or an exosome-associated marker in the sample obtained from the subject;
wherein an abnormal status of exosomes and/or the exosome-associated marker in the sample indicates the presence of the disease condition in the subject.

28. The method of claim 27 wherein sorting the labeled extracellular vesicles includes sorting them by parent cell type.

29. The method of claim 27 wherein the exosome or the exosome-associated marker in the sample is characterized by a specific miRNA marker.

30. The method of claim 27 wherein status is determined by determining the level of an indicator exosome or an exosome-associated marker in the sample.

31. The method of claim 30 wherein status is determined by comparing the level of the indicator exosome or an exosome-associated marker with the aggregate level of all exosomes in the sample.

32. The method of claim 27 wherein the level or presence of a suitable biomarker is determined in step c), and
d) the status of exosomes and/or an exosome-associated marker in the sample obtained from the subject is determined by comparing the level or presence of the biomarker determined in step c) with one or more reference values.

33. The method of claim 32 wherein the suitable biomarker is a specific miRNA marker or miRNA expression profile.

34. The method of claim 32 wherein the reference value is the level or presence of the same biomarker of step c) in samples obtained from a subject different from than the subject of step a).

35. The method of claim 27 wherein the method is used for screening for cancer in the subject.

36. The method of claim 27 wherein the method is used for screening for a specific disease condition other than cancer.

37. The method of claim 36 wherein the method is used for screening for a specific disease condition indicative of inflammatory disease, infectious disease, an autoimmune disease, hypersensitivity-associated inflammation, graft rejection, injury, disorder of apoptosis or disease associated with activation of the necroptosis activation pathway.

38. A method for in vitro screening for the presence of a disease condition in a test subject comprising
a) providing a biological sample obtained from the test subject,
b) isolating extracellular vesicles from said sample, wherein this step of isolating extracellular vesicles comprises
1) labeling the extracellular vesicles using fluorescence in situ hybridization, and
2) sorting the labeled extracellular vesicles by high resolution flow cytometry;
c) determining, from the extracellular vesicles isolated in step b), the status of exosomes and/or an exosome-associated marker in a sample obtained from the subject;
d) comparing the status of exosomes and/or an exosome-associated marker in the sample with the status of exosomes and/or the exosome-associated marker to the status of exosomes and/or an exosome-associated marker determined in a population of training subjects known to have the disease condition; wherein a similarity of the status of exosomes and/or the exosome-associated marker of the test subject to the status of exosomes and/or the exosome-associated marker of the training subjects indicates the presence of the disease condition in the subject.

39. The method of claim 38 wherein sorting the labeled extracellular vesicles includes sorting them by parent cell type.

40. The method of claim 38 wherein the level or presence of a suitable biomarker is determined in step c), and
d) the status of exosomes and/or an exosome-associated marker in the sample obtained from the subject is determined by comparing the level or presence of the biomarker determined in step c) with one or more reference values of the biomarker determined in the population of the training subjects.

41. The method of claim 38 wherein the suitable biomarker is a specific miRNA marker or miRNA expression profile.

42. The method of claim 38 wherein the method is used for screening for cancer in the subject.

43. The method of claim 38 wherein the method is used for screening for a specific disease condition other than cancer.

44. The method of claim 43 wherein the method is used for screening for a specific disease condition indicative of inflammatory disease, infectious disease, an autoimmune disease, hypersensitivity-associated inflammation, graft rejection, injury, disorder of apoptosis or disease associated with activation of the necroptosis activation pathway.
